(19) United States
(12) Patent Application Publication
(10) Pub. No.: US 2006/0110459 A1
(43) Pub. Date: May 25, 2006

(54) Triple natural polymer viscoelastic composition

(76) Inventors: Masoud R. Jafari, Arlington, TX (US); Kerry L. Markwardt, Burleson, TX (US); Alan L. Weiner, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 11/284,874

(22) Filed: Nov. 22, 2005

Related U.S. Application Data
(60) Provisional application No. 60/630,584, filed on Nov. 23, 2004.

(51) Int. Cl. A61K 31/737; A61K 31/728; A61K 31/7008; A61K 9/14

(52) U.S. Cl. 424/488; 514/54; 514/62

(57) ABSTRACT

A triple natural polymer viscoelastic composition is disclosed. By combining glucosamine sulfate (“GS”), sodium hyaluronate (“HA”) and chondroitin sulfate (“CS”), the triple natural polymer viscoelastic compositions of this invention can provide a viscoelastic agent at a surgical site that not only serves as a protective agent to ocular tissues, but that can also act as an agent to alleviate pain and inflammation associated with ocular surgery. Embodiments of this invention can comprise GS combined with existing HA/CS viscoelastic agents. Embodiments of this invention can also comprise GS and CS in combination with an irrigation solution. Further, to enhance retention time at the site of an intra-articulate application of an embodiment of this invention, the following biodegradable polymers may also be included in an embodiment of the compositions of the present invention: cellulose derivatives (e.g., hydroxypropylmethylcellulose, ethylcellulose, caboxymethylcellulose, etc.), carbopol, citosan, and collagen.

This application claims priority under 35 U.S.C. §119 to U.S. Provisional Patent Application No. 60/630,584 filed Nov. 23, 2004, the entire contents of which are incorporated herein by reference.

### TECHNICAL FIELD OF THE INVENTION

The present invention relates to novel viscoelastic compositions and their use in the field of surgery utilizing viscous and/or viscoelastic materials, also known as viscosurgery. In particular, the invention involves the combination of polymeric materials in aqueous solutions to enhance the performance of the viscosurgical materials, especially in certain environments. The invention also relates to methods of using such enhanced viscoelastic materials and similarly enhanced irrigation solutions for all conventional purposes, and particularly those in which retention of the viscoelastic material is desirable, such as in intra-articular use and in certain ophthalmic surgical procedures.

### BACKGROUND OF THE INVENTION

Viscous or viscoelastic agents used in surgery may perform a number of different functions, including, without limitation, maintenance and support of soft tissue, tissue manipulation, lubrication, tissue protection, and adhesion prevention. It is recognized that the differing rheological properties of these agents necessarily impact their ability to perform these functions, and, as a result, their suitability for certain surgical procedures. See, for example, U.S. Pat. No. 5,273,056, the contents of which are by this reference incorporated herein.

A number of viscous or viscoelastic agents (hereinafter “agents” or “viscoelastics”) are known for ophthalmic surgical use: Viscoat® (Alcon Laboratories, Inc.), which contains sodium hyaluronate and chondroitin sulfate; Provisc® (Alcon), Healon®, Healon® GV, and Healon®5 (Pharmacia Corporation), Amvisc® and Amvisc® Plus (Bausch & Lomb, Inc.), and Vitrax® (Allergan Inc.), all of which contain sodium hyaluronate; and Cellugel® (Alcon), which contains hydroxypropylmethylcellulose (HPMC). All of the foregoing examples of viscoelastics may be used in cataract surgery. They are used by the skilled ophthalmic surgeon for several purposes, including maintenance of the anterior chamber of the eye and protection of ophthalmic tissues during surgery, particularly corneal endothelial cells, and as an aid in manipulating ophthalmic tissues.

The use of viscoelastic compositions in ocular surgery is extensive. While all of the agents described above may be used during cataract, or other, ocular surgery, each has certain recognized advantages and disadvantages. For example, prior-art viscoelastic compositions are primarily intended to be used for their beneficial effects during an ocular surgery. However, post-surgical complications of ocular surgeries are well documented, and mainly include inflammation and pain. The inflammation associated with ocular surgeries is currently treated by surgeons through the use of topical steroids pre- and post-surgically. Some of the disadvantages of this traditional practice include poor patient compliance, additional cost and unwarranted steroid side-effects. Similarly, post-surgical pain is treated in a conventional manner, such as by oral medication, with similar disadvantages.

The use of viscoelastic agents in joint therapy is also known in the art. Viscoelastic joint therapy involves the intra-articular application of commercially available sodium hyaluronate viscoelastic materials such as HYLAN G-F 20, SYNVISC, HYALGAN, ARTZ, etc. These products are comprised of sodium hyaluronate (“HA”) in various molecular weights. The HA is thought to affect the rheology of the synovial fluid, producing an almost immediate sensation of free movement and a marked reduction of pain in patients suffering from chondromalacia and/or arthritis, and particularly osteoarthritis. A main disadvantage with these products, however, is that although HA is found in human (and other animal) bodies, and is present in relatively high amount in joint tissues and synovial fluid, HA is not the major substance within the intra-articular cartilage and, hence, it may not necessarily stop cartilage thinning and progression of arthritis. HA acts as a lubricant and shock absorber in joints.

Glucosamine sulfate (“GS”), however, is found largely in cartilage and plays an important role in its health and resiliency. As bodies age, they lose some of the GS and other substances in cartilage. This can eventually lead to the thinning of cartilage and the onset and progression of arthritis. GS very rapidly diffuses in most tissues and organs and it has a special attraction to articulate tissue (e.g., cartilage) and to bone. In several clinical studies, it has been shown that GS in oral dosage forms significantly eases osteoarthritis symptoms and its progression. It is also believed that GS may possess antioxidant ability and that it may have an inhibitory effect on prostaglandin synthesis.

Chondroitin sulfate (“CS”) is also known to be useful in the treatment of diseased or traumatized joints. See U.S. Pat. No. 5,498,606. CS further protects cartilage and surrounding joint tissues by acting as a coating agent. Studies have shown an increased benefit when combining GS and CS to treat intra-articular maladies. GS and CS both help to build cartilage and can help delay the release of enzymes that break down cartilage. Currently there are products comprised of GS and CS (or GS and CS alone) on the market as oral multivitamins or nutrients. These products have been shown clinically to be effective and safe to relieve osteoarthritis symptoms (e.g., pain, inflammation, and narrowing of joint space-width) and can be a great alternative to non-steroidal anti-inflammatory drugs (“NSAID”). However, the oral dosage forms of GS and CS have the disadvantages of poor absorption, delayed onset of action and short duration of action.

An injection dosage form (intra-articulate) of a combination of HA, GS and CS would have increased benefits over prior art polymeric combinations, and, in particular, over oral dosage forms, including the rapid onset of action, prolonged duration of action, and, more importantly, delivery of all three natural polymers into an affected joint. HA, GS and CS are naturally occurring polymers and are commercially abundant.

There is a need, therefore, for a composition of polymers, including a viscoelastic composition of polymers, that can not only provide the protective functions during surgery of prior art viscoelastic compositions, but that can also provide for relief of pain and inflammation associated with a surgery and for relief of intra-articulate maladies.

### SUMMARY OF THE INVENTION

The embodiments of the triple natural polymer viscoelastic compositions of this invention substantially meet these needs and others. The present invention is directed to improved viscoelastic compositions for performing surgery, especially ophthalmic surgery, and for performing therapies, especially viscoelastic joint therapy, by providing improved analgesic and anti-inflammatory properties. Embodiments of the compositions of this invention can comprise a combination of the natural and biocompatible polymers HA, GS and CS to form a viscous and elastic sterile solution that can be used as an intraocular and/or intra-articular agent.

The embodiments of the triple natural polymer viscoelastic compositions of the present invention are capable of several functions. When used for intraocular surgery, they can provide protection, as a traditional viscoelastic agent might, to the endothelial cells based on their viscoelastic characteristics. More importantly for the purposes of this invention, they can also provide anti-inflammatory and analgesic action towards control of pain and inflammation related to ocular surgery. When used as an intra-articulate agent, the embodiments of this invention can provide improved relief of osteoarthritis symptoms.

Post-surgical complications of ocular surgeries are well documented, including inflammation and pain. The use of viscoelastic agents to provide protection for the interior portions of the eye during ocular surgery is extensive. By combining GS, HA and CS, the embodiments of the triple natural polymer viscoelastic compositions of this invention can provide a viscoelastic agent at a surgical site that not only serves as a protective agent to the ocular tissues, but that can also act as an agent to alleviate pain and inflammation associated with ocular surgery. Embodiments of the compositions of this invention can also comprise GS and CS in combination with existing irrigation solutions to provide an anti-inflammatory effect. As an intra-articulate agent, the embodiments of this invention can provide benefits to a patient by replacing the diminished hyaluronan in joint tissues and diminished glucosamine in cartilage to prevent progression of osteoarthritis.

Embodiments of this invention can comprise GS combined with existing HA/CS viscoelastic agents, such as the Discovisc™ and Viscoat™ viscoelastic products manufactured by Alcon Laboratories, Inc. of Fort Worth, Tex. (“Alcon”). Embodiments of this invention can also comprise GS and CS in combination with an irrigation solution such as BSS™, BSS-Plus™ and StabIEyz™, all manufactured by Alcon. Further, to enhance retention time at the site of an intra-articulate application of an embodiment of this invention, the following biodegradable polymers may also be included in an embodiment of the compositions of the present invention: cellulose derivatives (e.g., hydroxypropylmethylcellulose (“HPMC”), ethylcellulose, caboxymethylcellulose, etc.), carbopol, citosan, and collagen.

The embodiments of the triple natural polymer viscoelastic compositions of this invention can provide the advantage of relief from the pain and inflammation associated with ocular surgery (e.g., cataract or vitreo-retinal surgery). Further, they can reduce or eliminate the need for topical application of steroid anti-inflammatory products during or after ocular surgery, which can result in better patient compliance and reduced side-effects. For intra-articulate applications, the benefits of the present-invention include combining the benefits of the three natural polymers HA, GS and CS into a single injectable formulation that can promote cartilage formation and repair and alleviate osteoarthritis symptoms. Further, such an injectable formulation provides for on-site delivery of the agent, with improved onset of action and duration of action.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1 shows the viscosity profile of various compositions of this invention plotted against the viscosity profile of a test solution.

### DETAILED DESCRIPTION OF THE PRESENT INVENTION

While particularly important in ophthalmic surgery, and especially cataract surgery, the methods and compositions of the present invention may be utilized in any viscosurgical procedure with a hyaluronate-based viscoelastic. In cataract surgery, the anterior chamber of the eye, i.e., the space between the iris and the corneal endothelium is filled with viscoelastic. The viscoelastic serves two purposes: (1) maintaining the corneal dome to give the surgeon an unobstructed view of the interior surgical site, and (2) protecting the delicate endothelial cells of the cornea by coating them. As discussed above, prior-art viscoelastic agents do not provide analgesic or anti-inflammatory effects, with the result that topical agents must be used to control pain and inflammation pre- and post-surgery. It would thus be preferable if the traditional viscoelastic functions and the anti-pain and anti-inflammation functions could be served by a single viscoelastic material. That objective is met using the methods and compositions of the present invention. The various embodiments of the viscoelastic compositions of this invention are also well suited for use as vitreous replacements in, for example, a vitreo-retinal surgery, and such use is contemplated to be within the scope of this invention.

Because of their ability to achieve enhanced retention times when injected into the body, the viscoelastic compositions of the present invention are also well-suited for joint therapy through intra-articular injection. The effect of conventional hyaluronate is enhanced by the addition of GS and CS in accordance with the teachings of this invention. U.S. Pat. No. 5,498,606, the entire contents of which are by this reference incorporated herein, discloses the anti-inflammatory and cell protective effects observed upon intra-articular injection of chondroitin sulfate in horse joints. More recently, it has been suggested that the intra-articular injection of VISCOAT, which contains a mixture of sodium hyaluronate and chondroitin sulfate, may cause cartilage regeneration in the joints of patients suffering from grade I and grade II osteoarthritis. In that regard, the contents of commonly assigned U.S. patent application Ser. No. 10/082,743 are by this reference incorporated herein. It is further contemplated that the embodiments of the triple natural polymer viscoelastic composition of the present invention can be configured to take advantage of the dilution resistant properties of the dilution resistant viscoelastic compositions disclosed in pending U.S. patent application Ser. No. 10/882,901, the contents of which are hereby fully incorporated by reference.

The term “hyaluronate-based viscoelastic” as used herein means any aqueous solution of hyaluronic acid or physiologically acceptable salts thereof, which is free of any significant amount of any low molecular weight, non-HA polymer. With the exception of Viscoat®, all of the commercial HA products described above are considered hyaluronate-based viscoelastics. Hyaluronate-based viscoelastics suitable for combining with GS and CS in accordance with the teachings of this invention include those that can generally be characterized as containing sodium hyaluronate (or other physiologically acceptable hyaluronate salt) having average molecular weights greater than 500,000 Daltons, preferably from about 1,000,000 to about 5,000,000 Daltons, and concentrations from about 1.0 to about 3.0% by weight.

Irrigating solutions suitable for combining with GS and CS to produce the embodiments of the polymeric irrigating solution in accordance with the teachings of this invention include any sterile, aqueous irrigating solution suitable for surgery. Preferred are balanced salt solutions such as BSS® or BSS PLUS® (Alcon Laboratories, Inc., Fort Worth, Tex.). The addition of polymers to the irrigating solution may be effected in the manner described in U.S. Pat. No. 5,409,904, hereby fully incorporated by reference. The relatively low weight CS suitable for purposes of the present invention would include material having an average molecular weight of less than about 100,000 Daltons, preferably from about 20,000 to about 80,000 Daltons, and most preferably from about 30,000 to about 50,000. Concentration ranges for the polymeric components (GS and CS) of the embodiments of the polymeric irrigating composition of the present invention will vary depending upon the molecular weight of the polymeric component chosen, but should be maintained at levels low enough to retain the flow properties desired for an irrigating solution. For CS, the concentration in the irrigating solution may be from 0.1 to 10% by weight, preferably from 0.5 to about 7%, and most preferably from about 2% to about 5% by weight. For GS, the concentration in the irrigating solution may be from 1% to 5%. For intra-articular use, the polymeric compositions of the present invention are mixed without an irrigation solution. The polymers can be mixed with a hyaluronate-based viscoelastic, as discussed below, to achieve the properties described herein.

The following examples are provided to further illustrate various features of the embodiments of the triple natural polymer viscoelastic composition of the present invention. The formulations listed below in Table 1 were prepared and tested for rheology and CDI (Cohesivity/Dispersivity Index) and compared to the viscoelastic VISCOAT®. Table 1 shows the composition for each formulation tested.

TABLE 1

| Batch No. | FID | Composition (w/v %) |
|---|---|---|
| 03-34322 | 105620 | GS 1% + HA 3% + CS 4% in PBS |
| 03-34461 | 105708 | GS 2% + HA 3% + CS 4% in PBS |
| 03-34504 | 105722 | GS 3% + HA 3% + CS 4% in PBS |
| 03-34689 | 105808 | GS 4% + HA 3% + CS 4% in PBS |
| Lot # 02H29A | VISCOAT ® | HA 3% + CS 4% in PBS |

GS (glucosamine sulfate),
HA (hyaluronic acid),
CS (chondroitin sulfate),
PBS (phosphate buffer saline)

The pH and osmolality for each formulation listed in Table 1 was measured and is presented in TABLE 2, below.

TABLE 2

| Batch No. | pH | Osmolality (mOsm/kg) |
|---|---|---|
| 03-34322 | 6.6 | 330 |
| 03-34461 | 6.2 | 352 |
| 03-34504 | 6.2 | 307 |
| 03-34689 | 6.4 | 359 |

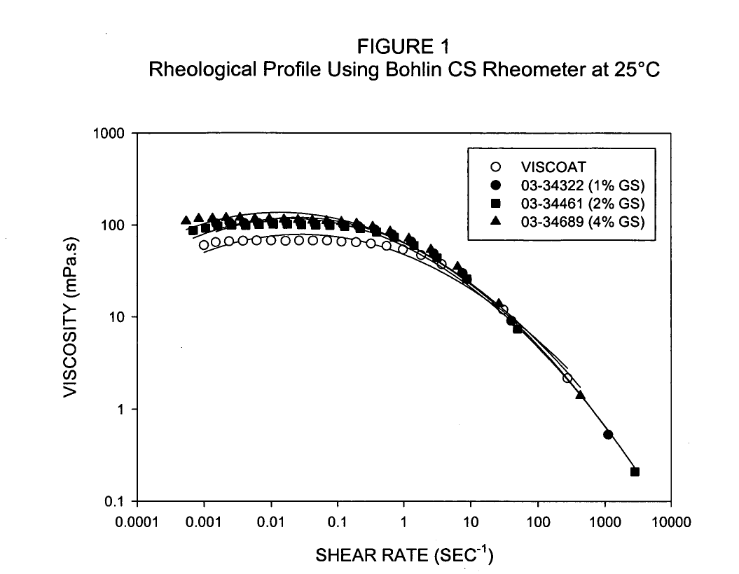

Rheological evaluation of each formulation of Table 1 was also conducted in comparison to VISCOAT®. FIG. 1 is a graph showing the Theological profile of each formulation from Table 1, as well as the Theological profile for VISCOAT®. The viscosity data for each formulation of Table 1 at various shear rates are presented in TABLE 3, below.

TABLE 3
Average Viscosity at various shear rates (Bohlin CS Rheometer at 25° C.)

| | Viscosity (Pa . s) | | | | | |
|---|---|---|---|---|---|---|
| Batch No. | Zero Shear | 0.1 | 1 | 2 | 10 | 100 |
| 03-34322 | 106 | 102 | 70 | 58 | 25 | 10 |
| 03-34461 | 102 | 97 | 68 | 58 | 23 | 5 |
| 03-34504 | 138 | 130 | 95 | 70 | 25 | 5 |
| 03-34689 | 102 | 98 | 63 | 55 | 25 | 5 |
| VISCOAT | 68 | 66 | 54 | 44 | 22 | 9 |

Sample Preparation and Determination of Viscosity

The formulations listed in Table 1 were prepared as follows. The plunger was removed from a 10 mL sterile plastic syringe (A1) and the other end closed off with a sterile tip cap. The syringe A1 was placed upright in a beaker on a balance. An appropriate amount of sterile HA polymer was weighed and transferred into the syringe A1. In a similar fashion, an appropriate amount of sterile GS and CS were weighed and transferred into a syringe B1. Throughout this description, “appropriate amount” and “sufficient quantity” mean an amount of a material, polymer or solution necessary to provide one of the compositions listed in Table 1. Note that other syringe sizes can also be used, as appropriate.

Formulation buffer solution was prepared and sterile filtered through a 0.2 micron filter (other filter sizes may be used, as appropriate). In a similar fashion to that described above, a sufficient quantity of sterile buffer solution was added to two other syringes A2 and B2. The plungers were then carefully placed back into each individual syringe. The tip caps were removed from syringes A1 and A2 and the two syringes were connected together via a Luer-Lock connector. The contents of syringes A1 and A2 were then thoroughly mixed by alternately pushing plungers of the conjoined syringes until a homogenous mixture was obtained. The final content of the mixed solution was collected into one of the syringes, e.g., syringe A1.

In a similar manner, the tip caps were removed from syringes B13 and B2 and the two syringes were connected together via a Luer-Lock connector. The contents of syringes B1 and B2 were then thoroughly mixed by alternately pushing plungers of the conjoined syringes until a homogenous mixture was obtained. The final content of the mixed solution was collected into one of the syringes, e.g., syringe B1.

Syringes A1 and B13 were then connected via a Luer-Lock connector and their contents were then thoroughly mixed by alternately pushing plungers of the conjoined syringes until a homogenous mixture was obtained. The resulting composition is then stored overnight in a refrigerator to obtain a complete hydration. This final formulation is a sterile, homogenous, clear polymer mixture solution. The Theological profile of a sample of each such formulation was then determined the following day.

Determination of Rheological Profile (Zero Shear Viscosity)

The rheological profile was determined by using a Bohlin CS Rheometer. A 4° cone and 40 mm diameter plate (CP 4/40) at a gap width of 0.15 mm was used. Viscosity was determined at 25° C. Shear stresses applied were from 0.06 to 139 Pa. The corresponding shear rate and viscosity was calculated by the Bohlin software after 200 seconds of integration or whenever the system approved steady state was reached.

Viscosity Results

Based on the applied shear stress, the Bohlin CS Rheometer calculates the shear rate and apparent viscosity at that shear rate. The logarithm of viscosity in Pascal seconds (Pas) is plotted on the Y-axis against the corresponding shear rate in reciprocal seconds (1/s) on the X-axis. Usually, for most viscoelastics, there is a significant plateau for viscosity, at low shear rates. The extent of the plateau varies with different viscoelastics. The intercept of the plateau on the Y-axis is considered as zero shear viscosity. When the shear rate is increased further, viscosity drops exponentially. The viscosity profile for each formulation of Table 1 is plotted in FIG. 1.

Rheological evaluation of the formulations of the present invention, as listed in Table 1, clearly show that by addition of GS to each formulation, the viscoelastic (viscous and elastic) properties of each triple natural polymer viscoelastic composition are maintained and are similar to those of VISCOAT®. The addition of GS in 1 to 4% w/v has no negative impact in the rheological properties of a viscoelastic such as VISCOAT®. A preferred formulation of the triple natural polymer viscoelastic composition of this invention for intra-ocular and intra-articulate applications can thus comprise the following:

| | | |
|---|---|---|
| Na Hyaluronate (HA) | 3% | w/v |
| Chondroitin Sulfate (CS) | 4% | w/v |
| Glucosamine Sulfate (GS) | 1 to 4% | w/v |
| Sodium Phosphate Monobasic | 0.045% | w/v |
| Sodium Phosphate Dibasic | 0.2% | w/v |
| NaCl | 0.43% | w/v |
| Water-for-injection | QS 100% | |

Cohesivity and dispersivity of each of the formulations of this invention listed in Table 1 were also evaluated, as these values closely relate to the retention of the formulation at the site of application. TABLE 4, below, lists the CDI for each formation as compared to VISCOAT®. Addition of GS appears to enhance the dispersivity of a VISCOAT® formulation, and thus, the embodiments of the triple natural polymer formulations of this invention are likely to possess longer retention than VISCOAT®, which would be favorable for intra-articulate application. For intra-ocular applications, higher concentration of GS (e.g., 4%=CDI 12.1) may be preferred for higher cohesivity and ease of removal.

TABLE 4
Formulation CDI

| Batch No. | CDI |
|---|---|
| 03-34322 | 3.3 |
| 03-34461 | 2.9 |
| 03-34504 | 2 |
| 03-34689 | 12.1 |
| VISCOAT | 8 |

Embodiments of the present invention can comprise methods of introducing or instilling the embodiments of the triple natural polymer viscoelastic composition and/or the embodiments of the polymeric irrigating solution of the present invention into a therapy site (e.g., a joint or eye), for example, to perform intra-articular therapy or to perform ophthalmic surgery.

The present invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims.

What is claimed:

1. A triple natural polymer viscoelastic composition, comprising:
a hyaluronate-based viscoelastic agent;
glucosamine sulfate; and
chondroitin sulfate.

2. The composition of claim 1, wherein the hyaluronate-based viscoelastic is an aqueous solution sodium hyaluronate having an average molecular weight greater than 750,000 Daltons and a concentration by weight from 1% to 3%.

3. The composition of claim 1, wherein the chondroitin sulfate is present at a concentration by weight from about 0.1 to about 7%.

4. The composition of claim 3, wherein the concentration of the chondroitin sulfate is about 2% by weight and the chondroitin sulfate has an average molecular weight of about 20,000 to about 80,000 Daltons.

5. The composition of claim 3, wherein the concentration of the chondroitin sulfate is about 4% by weight and the chondroitin sulfate has an average molecular weight of about 20,000 to about 80,000 Daltons.

6. The composition of claim 1, wherein the glucosamine sulfate is present at a concentration by weight from about 1% to about 5.0%.

7. The composition of claim 6, wherein the glucosamine sulfate is present at a concentration by weight of about 4%.

8. The composition of claim 1, wherein the chondroitin sulfate is present at a concentration by weight of about 4%., the glucosamine sulfate is present at a concentration by weight of about 1% to about 4%, and the hyaluronate-based viscoelastic is an aqueous solution sodium hyaluronate having a concentration by weight of about 3%.

9. The composition of claim 1, wherein the composition is used as a vitreous replacement in a vitreo-retinal surgery.

10. The composition of claim 1, wherein the composition is administered through an intra-articular injection for intra-articular therapy.

11. A method of performing intra-articular therapy comprising: introducing a triple natural polymer viscoelastic composition into a therapy site, wherein the triple natural polymer viscoelastic composition comprises:
a hyaluronate-based viscoelastic agent;
glucosamine sulfate; and
chondroitin sulfate.

12. The method of claim 11, wherein the hyaluronate-based viscoelastic is an aqueous solution sodium hyaluronate having an average molecular weight greater than 750,000 Daltons and a concentration by weight from 1% to 3%.

13. The method of claim 11, wherein the chondroitin sulfate is present at a concentration by weight from about 0.1 to about 7%.

14. The method of claim 13, wherein the concentration of the chondroitin sulfate is about 2% by weight and the chondroitin sulfate has an average molecular weight of about 20,000 to about 80,000 Daltons.

15. The method of claim 13, wherein the concentration of the chondroitin sulfate is about 4% by weight and the chondroitin sulfate has an average molecular weight of about 20,000 to about 80,000 Daltons.

16. The method of claim 11, wherein the glucosamine sulfate is present at a concentration by weight from about 1% to about 5.0%.

17. The method of claim 16, wherein the glucosamine sulfate is present at a concentration by weight of about 4%.

18. The method of claim 11, wherein the chondroitin sulfate is present at a concentration by weight of about 4%., the glucosamine sulfate is present at a concentration by weight of about 1% to about 4%, and the hyaluronate-based viscoelastic is an aqueous solution sodium hyaluronate having a concentration by weight of about 3%.

19. The method of claim 11, wherein the composition is introduced to the therapy site by an intra-articular injection.

20. A polymeric irrigating composition, comprising:
an aqueous irrigating solution;
glucosamine sulfate; and
chondroitin sulfate.

21. The composition of claim 20, wherein the aqueous irrigating solution is a sterile aqueous irrigating solution selected from the group consisting of BSS and BSS Plus.

22. The composition of claim 20, wherein the chondroitin sulfate is present at a concentration by weight from about 0.1 to about 7%.

23. The composition of claim 22, wherein the concentration of the chondroitin sulfate is from about 2% to about 5% by weight and the chondroitin sulfate has an average molecular weight of about 20,000 to about 80,000 Daltons.

24. The composition of claim 20, wherein the glucosamine sulfate is present at a concentration by weight from about 1% to about 5.0%.

25. The composition of claim 20, wherein the composition is used as an irrigating solution in an ophthalmic surgery.

26. A method of performing ophthalmic surgery, comprising instilling in the eye a polymeric irrigating composition, wherein the polymeric irrigating solution comprises:
an aqueous irrigating solution;
glucosamine sulfate; and
chondroitin sulfate.

27. The method of claim 26, wherein the aqueous irrigating solution is a sterile aqueous irrigating solution selected from the group consisting of BSS and BSS Plus.

28. The method of claim 26, wherein the chondroitin sulfate is present at a concentration by weight from about 0.1 to about 7%.

29. The method of claim 28, wherein the concentration of the chondroitin sulfate is from about 2% to about 5% by weight and the chondroitin sulfate has an average molecular weight of about 20,000 to about 80,000 Daltons.

30. The method of claim 26, wherein the glucosamine sulfate is present at a concentration by weight from about 1% to about 5.0%.
